(19) United States
(12) Patent Application Publication
(10) Pub. No.: US 2026/0159542 A1
(43) Pub. Date: Jun. 11, 2026

(54) SEPARATION OF BIOMOLECULES BY MEANS OF AFFINITY CHROMATOGRAPHY

(71) Applicant: i3 Membrane GmbH, Hamburg (DE)

(72) Inventors: Kenneth RENFREW, Huntersville, NC (US); Stephan BRINKE-SEIFERTH, Hamburg (DE)

(73) Assignee: i3 Membrane GmbH, Hamburg (DE)

(21) Appl. No.: 18/722,870

(22) PCT Filed: May 13, 2023
(86) PCT No.: PCT/EP2023/062861

§ 371 Date: Jun. 21, 2024

(30) Foreign Application Priority Data
Dec. 30, 2022 (DE) 102022135036.3
Apr. 18, 2023 (DE) 102023109773.3

(51) Int. Cl. C07K 1/22; B01D 15/38; B01D 15/42; B01J 20/28; B01J 20/285; C07K 1/24

(52) U.S. Cl. CPC C07K 1/22; B01D 15/3809; B01D 15/3885; B01D 15/424; B01J 20/28033; B01J 20/285; C07K 1/24

(57) ABSTRACT

The Invention relates to a method for purifying, separating, selecting and/or isolating biomolecules by means of affinity chromatography, wherein a stationary phase containing, in particular selective, binding sites formed by ligands and/or receptors is provided.

There is a need to provide a suitable method for purifying, separating, selecting and/or isolating biomolecules in which desorption via change of the pH value, in particular using acidic or alkalic buffer solutions, and or via application of salt solutions is avoided, thus eliminating potential harm of degradation of the separated biomolecules, which especially, if degraded by any means, could provide health risks in medical applications or alike.

This might be solved by applying a voltage between two electrodes, in particular with the stationary phase placed between those electrodes.

### TECHNICAL FIELD

The Invention relates to a method for purifying, separating, selecting and/or isolating biomolecules by means of affinity chromatography, wherein a stationary phase containing, in particular selective, binding sites formed by ligands and/or receptors is provided. Biomolecules brought in contact to the stationary phase may bind to the, in particular selective, binding sites by forming a ligand/receptor complex. Subsequently, elution of said biomolecules previously bound is performed.

### BACKGROUND ART

Generally, biomolecules are sought-after species for medical applications, e.g., proteins to be used as drugs or alike. As such, the desired biomolecules are to be produced with high purity, thus need to be separated from impurities resulting from the formation of those biomolecules. Such separation is often performed by bind-elute processes, in particular using affinity chromatography.

As such, affinity chromatography is a method for separating biomolecules based on a highly selective macromolecular binding interaction between the biomolecule and a complementary substance.

Affinity chromatography is based on specific binding interactions between an analyte of interest (normally dissolved in a mobile phase, thus some fluid), and a binding partner, usually in form of a ligand or receptor (immobilized on the stationary phase). Within typical affinity chromatography applications, the ligand is attached to any solid, insoluble material—usually a polymer—which is particularly chemically modified to introduce reactive functional groups with which the ligand or receptor can react, forming stable covalent bonds. The stationary phase is usually loaded into a column to which the mobile phase, usually in form of fluids containing a target biomolecule, is introduced. Biomolecules with high binding affinity to the ligand/receptor will bind to the ligand/receptor and will subsequently remain bound to the stationary phase. Afterwards the column is rinsed to remove any unwanted, thus non-target biomolecules by disrupting their weaker interactions with the stationary phase, while the biomolecules of interest will remain bound. Target biomolecules may then be removed by applying a so-called elution buffer, which disrupts interactions between the bound target biomolecules and the ligand. The target molecule is thus recovered in the eluting solution. Usually, such elution buffers are fluids with pH values in the acidic or alkalic regime. For example, a common elution buffer to be used is an acetate buffer solution, e.g., at pH 3. Alternatively, or additionally, salt solution may also be used for elution. Both of those elution methods, may lead to harsh conditions which may be harmful for any biomolecule, e.g., may lead to agglomeration, fragmentation or denaturation of the desired species, especially with respect to proteins.

Other methods for separating biomolecules in general comprise filtration via electro-sorption methods. Within those, EP 3 115 099 B1 describes a method for enhancing retention of biomolecules with a metal coated polymer membrane by applying a voltage to the membrane. Moreover, WO 2021/084080 A1 describes a method for electro-desorption of molecules adsorbed to a charged membrane with a metal coating by applying a voltage with opposite polarity to the charge of the membrane to the coating.

Generally, biomolecules like proteins are amphoteric species, which usually leads to those biomolecules having an overall charge different form neutral at certain pH values. Hence, generally, such biomolecules may be bound to a charged surface by means of electro-desorption. However, there is usually no selectivity for specific biomolecules provided, since generally each all biomolecules with charges of the same polarity are all absorbed or none are absorbed, further harsh conditions might be required.

Based on this, there is a need to provide a suitable method for purifying, separating, selecting and/or isolating biomolecules in which desorption via change of the pH value, in particular using acidic or alkalic buffer solutions, and or via application of salt solutions is avoided, thus eliminating potential harm of degradation of the separated biomolecules, which especially, if degraded by any means, could provide health risks in medical applications or alike.

This might be solved by applying a voltage between two electrodes, in particular with the stationary phase placed between those electrodes.

### SUMMARY OF THE INVENTION

This task is solved by a method for purifying, separating, selecting and/or isolating biomolecules by means of affinity chromatography comprising the following steps: a) providing a stationary phase, wherein the stationary phase contains, in particular selective, binding sites formed by ligands and/or receptors; b) bringing a first fluid containing biomolecules in contact with the stationary phase, thereby binding at least parts of the biomolecules to the, in particular selective, binding sites of the stationary phase by forming a ligand/receptor complex, in particular via non-covalent interaction(s), wherein one of the biomolecules and binding sites acts as the ligand and the other of the biomolecules and binding sites acts as the receptor; c) preferably passing at least parts of the first fluid over and/or through stationary phase and/or optionally bringing a second fluid, in particular without biomolecules, in contact with the stationary phase; d) eluting biomolecules previously bound in step b); characterized in that elution in step d) is performed by applying a voltage between two electrodes and an affinity chromatography device for purifying, separating, selecting and/or isolating biomolecules comprising a stationary phase characterized in that the stationary phase comprises, in particular is formed by, at least one porous affinity substrate arranged between at least two electrodes, particularly in a stacked arrangement, wherein the at least one affinity substrate comprises at least one substrate as stationary phase with binding sites formed by ligands and/or receptors immobilized on the porous affinity substrate.

Within the method according to the invention, a stationary phase is provided for purifying, separating, selecting and/or isolating of biomolecules by means of affinity chromatography, in particular a bind-elute process. Provision of such stationary phase is referred to as “step a)” of the disclosed method. Said stationary phase contains, in particular selective, binding sites formed by ligands and/or receptors. Preferably, only either ligands or receptors form the binding sites. Preferably the ligands and/or receptors are of a single type, in particular belong, e.g., to a single protein class. Suitable stationary phases may include but are not limited to such formed by, e.g., gels, resins or glass fibers but may also be provided by any suitable porous substrate(s), e.g., membranes, in particular polymeric membranes, as base material of said stationary phase. Any material based on which a suitable stationary phase may be formed must at least be able to host, in particular selective, binding sites formed by ligands and/or receptors, in particular with those binding sites being accessible to the biomolecules to be purified, separated, selected and/or isolated using the method disclosed, e.g., by bringing the stationary phase in contact with fluids containing those biomolecules. For this purpose, those ligands and/or receptors forming the binding sites are preferably immobilized on the base material of the stationary phase. Preferably, the stationary phase is porous, in particular permeable, in particular to fluids, e.g., water and/or alcohol, and the ligands and/or receptors forming the binding sites are preferably immobilized on an inner and/or outer surface of the stationary phase. In this regard, an inner surface is in particular a surface located within open pores of the stationary phase. The inner surface(s) is/are preferably accessible to fluids from the outside.

Most preferably, the stationary phase is formed by at least one porous substrate, in particular a membrane, preferably polymeric membrane, with the ligands and/or receptors forming the binding sites being in particular immobilized on an inner and/or outer surface of the at least one porous substrate in particular membrane. Consequently, the ligands and/or receptors are in particular located on an inside of the porous substrate, in particular membrane, in particular open pores of the porous substrate, in particular membrane, and/or on an outside of the porous substrate, in particular membrane. Hereafter, porous substrate, in particular membranes, hosting and/or containing and/or carrying ligands and/or receptors and/or on which the ligands and/or receptors are immobilized on are referred to as “affinity substrates, in particular membranes”.

In particular, instead of a single stationary phase, at least two stationary phases may be provided, which in particular may be or may not be stationary phases formed equally, e.g., contain the same ligands and/or receptors. In particular, using more than one stationary phase may enhance binding capacity of the stationary phase(s) in total since more ligand and/or receptor may be provided.

Within the method according to the invention, a first fluid containing bio-molecules is brought in contact with the stationary phase as second step of the method, hereafter referred as “step b)”. Thereby, at least parts of the biomolecules are bound to the, in particular selective, binding sites of the stationary phase by forming a ligand/receptor complex, in particular via non-covalent interaction. Establishing such complexes is based on the overall sum of interactions forces between the biomolecules and the binding sites. Of those, the biomolecules and the binding site, one is acting as the ligand with the other acting as the corresponding receptor. An attractive overall sum of interaction forces may yield to a high binding affinity between the bio-molecules and respective binding sites. For achieving such attractive overall sum of interaction forces, such respective binding sites and the biomolecules must be complementary, e.g., by having complementary three-dimensional shape, in particular, e.g., by having complementary local spatial conformations or alike.

Preferably, the ligands and/or receptors used for forming the binding sites are antigen ligands and/or receptors. Those are preferably proteins or aptamers, in particular oligopeptides or oligonucleotides. Such may be preferably immobilized on the stationary phase, in particular surfaces, in particular outer and/or inner surfaces of, the stationary phase, in particular the at least one affinity substrate, in particular membrane. E.g., techniques for immobilizing such ligands and/or receptor include but are not limited to EDC or DCC coupling and/or radical coupling, in particular through electronic beam treatment.

In this regard, using EDC or DCC coupling for disposing the ligands and/or receptors forming the binding sites usually provides such with a fairly regular orientation of the binding sites on the surface(s), however usually in relative low amounts. Especially, EDC or DCC needs an initial grafting step to prepare the surface(s) of the stationary phase(s) for binding, e.g., forming amine-or carboxy-functions on those surfaces, to which ligands and/or receptors may be bound afterwards, e.g., using amination or alike. On the contrary, using radical coupling techniques provides a less extensive method to immobilize binding sites on the stationary phase(s). Usually, stationary phases hosting binding sites prepared by radical coupling techniques contain higher amounts of binding sites. However, said binding sites may be disposed in a rather irregular orientation.

The biomolecules to be purified, separated, selected and/or isolated may include but are not limited to proteins, in particular immunoglobulins, or antibodies, in particular, natural or monoclonal antibodies, or enzymes or nucleic acids, like RNA or DNA. In particular, the term “biomolecules” may incorporate viruses. In this regard, e.g., the ligand/receptor complex may be formed between a virus envelope protein and a complementary binding site. In particular the ligands/receptors are in particular chosen, such that the bindings sites provided by those ligands/receptors may efficiently and in particular selectively bind the biomolecules to be purified, separated, selected and/or isolated.

As an example, binding sites formed by antigens, may be used for purifying, separating, selecting and/or isolating antibodies. As such, e.g., protein A may be used for purifying, separating, selecting and/or isolating, especially human, immu-noglobulin G antibodies (IgG), in particular IgG1, IgG2 and IgG4, since there is a high binding affinity between those IgGs and protein A. In this regard, e.g., protein A as a ligand in particular binds to Fc receptors of IgGs. Therefore, Protein A might be used as binding site. As another example, protein G may be analogously used for purifying, separating, selecting and/or isolating IgG3. Moreover, as yet another example, DNA or RNA, in particular in form of different strands and/or sequences and/or lengths, may be purified, separated, selected and/or isolated using, e.g., corresponding adaptamers immobilized on the stationary phase.

In particular, within the method according to the invention, the first fluid may contain more than one type of biomolecule(s), thus at least a first biomolecule and a second biomolecule. E.g., the first fluid might be blood. As such the terms “first biomolecule” and “second biomolecule” do not refer to two biomolecules of the same type but refer to biomolecules of different type. For separating those, the first and second biomolecule, the ligands and/or receptors acting as binding sites are chosen, so that the first biomolecule has a higher binding affinity to the ligands and/or receptors compared to the second biomolecule, in particular compared to all other biomolecules contained in the first fluid, and/or so that the binding affinity of the second biomolecule, in particular all other biomolecules contained in the first fluid different form the first biomolecule, is/are low and/or zero. Consequently, selectivity is achieved due to pre-dominantly or solely binding the first biomolecule to the binding sites with the second and/or other biomolecule(s) not being bound to the binding sites provided.

While, to and/or after bringing the fluid in contact with the stationary phase and thereby binding the biomolecules to the binding sites in step b), preferably at least parts of the first fluid are passed through and/or over the stationary phase and in particular thereafter removed and optionally a second fluid, in particular without further biomolecules, is added, in particular brought in contact with the stationary phase (together referred to as, in particular optional, “step c)”). For removal a rinsing fluid might be used prior to bringing into contact the second fluid. Second fluid and rinsing fluid might be of same or different type.

According to the invention, after the binding of the biomolecules in step b) and in particular removal of the first fluid and addition of the second fluid or removal of at least parts of the first fluid in step c), elution of the biomolecules is commenced, hereafter referred to as “step d)”, in particular yielding an eluate containing eluted biomolecules. The eluate, preferably is comprised by the second fluid and eluted bio-molecules.

Said elution in step d) is performed by applying a voltage between two electrodes, particularly after bringing in contact the stationary phase with a rinsing fluid and/or after rinsing the stationary phase and/or while bringing in contact the stationary phase with the second fluid, while there is in particular no voltage applied prior to step d), in particular no voltage and/or no electric field over the stationary phase is applied within steps a) to c). Preferably, the electrodes are permeable, in particular to fluids, as well. In particular, the stationary phase is located between the electrodes, preferably there is not more than 5 cm, particularly not more than 1 cm distance between one or both electrodes and the stationary phase and/or between both electrodes. By applying a voltage between the electrodes, an electric field is induced/established in a vicinity of the stationary phase, particularly with the stationary phase located within the electric field, which may lead to interaction between the induced/established electric field and the biomolecule bound and/or the ligands/receptors forming the binding sites respectively. Applying such voltage, respectively the resulting electric field, respectively said interaction between the established electric field and the biomolecules bound and/or ligands/receptors forming the binding sites respectively may in particular reduce attractive forces or even introduce additionally repulsive forces between the biomolecule and the binding site. The electric field might be part of an electromagnetic field while an electric field generated by a DC voltage is preferred.

By increasing the voltage applied to a certain voltage, such repulsive forces, in particular the electric field induced/established by said voltage may be sufficient to switch the overall interaction force between the biomolecules and the stationary phase, in particular the binding sites from attractive to repulsive. Such effects may be based on slight changes in the conformations, in particular secondary, tertiary or quaternary, in particular protein, structures, of at least one of the biomolecules and/or binding sites, yielding a loss of those being complementary to each other, in particular due to interaction with the electric field induced/established by the voltage applied. However, such slight conformational changes usually do not yield to denaturation, agglomeration or alike of the biomolecules. In particular such conformational changes only exist for a limited time and are easily reversed, in particular automatically by the biomolecules losing proximity to the electromagnetic or electric field, e.g., by in particular removing the eluate from the field and/or stationary phase.

Preferably, the voltage applied is in the range from 0.1 to 50 volts, in particular from 0.1 to 3 volts, in particular 1.5 to 3.0 volts, in particular to 2.5 volts. In particular voltages exceeding 3 volts are solely applied for short amounts of time, in particular usually for less than 2 seconds. The voltage may in particular be either DC or AC, while DC or AC with frequencies below 10 Hz are preferred in general. Such voltage usually does not harm the biomolecules and in particular provides mild elution conditions.

Elution via applying a voltage eliminates the need of using common elution principles of eluting via pH value amendments or elution using, in particular highly concentrated, salt solution, both of those providing somewhat harsh conditions for the biomolecules which may be harmful for those. Thus, preferably, no elution by means of, in particular overall, pH-value amendment, e.g., by introduced fluids, is performed within the method according to the invention and/or all fluids added within and/or prior the elution step, in particular second fluids and/or rinsing fluids, have a pH value equal to the first fluid and/or differ in pH value by less than 1.0, in particular less than 0.5, in particular less than 0.2, in particular less than 0.1. Preferably, all fluids used possess pH value providing mild conditions for the biomolecules to be purified, separated, selected and/or isolated. As such, pH values, which may be considered “neutral”, are preferred, e.g., pH values between pH 6 and pH 8, especially between pH 7 and 7.8, most preferably approx. pH 7.4. In this regard, liquids, in particular all liquids, introduced within the method may be or comprise buffer solutions used for maintaining constant pH values and/or ions in small amounts and/or salts in small amounts, preferably below 150 mM. As such, those buffer solutions contain salts in small amounts with those amounts being low compared to concentrations of salt solutions commonly used for common salt elution. Especially, those buffer solutions, e.g., phosphate-buffered saline (PBS), may be in particular isotonic and thus provide osmolarity and ion concentrations which match those of the human body, which may be particularly beneficial for medical related applications. In this regard, commonly used PBS, rinsing fluid, second fluid and/or all fluids applied after binding and prior and while eluting may comprise salts concentrations of approximately 150 mM or less. However, using fluids (rinsing fluid, second fluid and/or all fluids), especially buffer solution, which lesser concentrations is preferred, e.g., in particular with salt concentrations usually being below 10 mM, in particular below 5 mM, in particular below 2 mM, in particular below 1 mM, in particular below 0.5 mM, preferably below 0.25 mM, most preferred approx. 0.15 mM. Hence, there are preferably no salt solutions with salt concentrations exceeding those typical buffer concentrations as depicted above added, in particular prior and/or within the elution step. Respectively fluids “free of salts” are used, in particular for elution of the bound biomolecules. In this context, liquids with salt ion concentrations being negligible-and in this regard buffer solutions with salt concentration matching those described-are considered “free of salts”. Preferably, phosphate-buffered saline (PBS), in particular PBS with salts concentrations below 10 mM, in particular below 5 mM, in particular below 2 mM, in particular below 1 mM, in particular below 0.5 mM, preferably below 0.25 mM, most preferred approx. 0.15 mM, may be used as buffer solution, particularly as second fluid, for maintaining pH within the range of pH 6 to pH 8, in particular pH 7.4, which is considered a solution “free of salt” within the meaning of the disclosure. PBS used in common concentrations usually provides isotonic conditions, in particular with osmolarity and ion concentrations which match those of the human body.

Using the method disclosed, e.g., within an exemplary study, protein A was used as a binding site for isolation and extraction of IgGs. In this regard, a solution of IgGs in a PBS buffer (pH 7.4) was used. By bringing said solution into contact with the stationary phase provided in form of a polymeric membrane containing protein A as binding sites, the IgGs were efficiently and selectively bound to those binding sites. Thereafter, elution was performed using further PBS buffer solution without further IgGs by applying a voltage of approx. 2.2 to 2.3 volts. The yielded eluate contained approximately 90% of the IgGs previously provided, in particular bound to the binding sites. The same method was repeated several times using the same stationary phase. Within each repetition approximately 90% of the IgGs provided/bound were regained via elution showing the reliability and durability of the disclosed method.

In particular, an eluate yielded in step d) containing eluted biomolecules may further be used in and/or subjected to further methods. Those further methods may include but are not limited to performing detection reactions, preparation methods, medical applications or alike, e.g., including manufacturing very high-purity drugs or active substances for cancer treatment or alike.

Preferably, at least one of the electrodes is, in particular the electrodes, preferably all electrodes, are, formed by a conductive, in particular metal, coating and/or metallic net disposed at least on one non-conductive carrier, in particular formed by polymeric membrane(s) and/or porous substrate(s), and/or disposed on the at least one affinity substrate, in particular membrane. Preferably, at least one affinity substrate, in particular membrane and/or the at least one non-conductive carrier may act as an insulator between the electrodes. Preferably, gold or platinum is used as material for electrodes and/or the conductive metal coating(s). Preferably, the metal used is pure and/or the coating or layer consists of solely a single metal. Especially, the affinity substrate, in particular membrane itself is non-conductive and only the coating is electrically conductive. Especially only one kind of conductive material and/or metal is used for the electrodes and/or conductive coatings. In particular, the conductive coating is preferably no (non-gold and non-platinum) metallic net coated with gold or platin, in particular preferably not formed by a metallic net.

In yet another embodiment, the stationary phase may contain at least one of the electrodes, in particular formed by metal coating(s) and/or metallic net(s) disposed on the at least one affinity substrate, in particular membrane, and/or on the at least one non-conductive carrier. The ligands and/or receptors forming the binding sites may be immobilized on the at least one electrode contained in the stationary phase, e.g., in particular including but not limited to by covalent or associative bonding of the receptor and/or ligand to the metal. In this regard, Sulfur-gold bonding may be used to for binding ligands and/or receptors to the electrode. In this regard, binding the ligands and/or receptors to the metal may be beneficial if merely a small number of binding sites is needed to perform the method at hand, in particular for analytical applications. However, for preparation methods, for which higher amounts of binding sites are usually desired, disposing binding sites on the substrate(s), in particular membrane(s), or disposing the binding sites on both the substrate(s), in particular membrane(s) and the electrode(s) is preferred, especially when multiple stationary phases are used and arranged together between two electrodes.

According to a beneficial embodiment, the method may be enhanced by using stationary phase which consists of at least two specific stationary phases, in particular each formed by at least one, preferably a multitude of, in particular up to 50, in particular between 5 and 20, preferably less than 10, specific porous substrate, in particular specific permeable membrane(s). Those specific stationary phases preferably contain specific binding sites formed by ligands and/or receptors. Of those, the specific binding sites of a first specific stationary phase are formed by first type of ligands and/or receptors and the specific binding sites of a second specific stationary phase are formed by second type of ligands and/or receptors. Especially, those first and second ligands and/or receptors may be different, especially ligands/receptors of different type, e.g., different protein classes or alike. Most preferably, the first and second ligands are chosen such that, for any two different biomolecule(s) to be separated, purified, selected and/or isolated and/or contained in the first fluid, those different biomolecule(s) each have a high binding affinity to solely one of the first and second type of ligands and/or receptors with binding affinity to the other of the first and second type of ligands and/or receptors being lower, low and/or zero.

As an example, one may consider a stationary phase consisting of two specific stationary phases A′ and B′. Specific stationary phase A′ contains ligands and/or receptors A# and specific stationary phase B′ contains ligands and/or receptors B#. By bringing a fluid containing biomolecules A and B in contact with the stationary phase, of those, biomolecule A has a high binding affinity to ligand/receptor A# and a low binding affinity to a ligand/receptor B# and biomolecule B has a low binding affinity to ligand/receptor A# and a high binding affinity to a ligand/receptor B#, biomolecule A is solely adsorbed to ligand/receptor A# and biomolecule B is solely adsorbed to ligand receptor B #. Therefore, both biomolecules A and B are selectively bound to solely one of the specific stationary phases A′ or B′.

In general, with respect to arranging the at least two, in particular different specific, stationary phases several beneficial arrangements may be used.

In particular the at least two, in particular different specific, stationary phases may be arranged such that the first and second, in particular different specific, stationary phases, in particular all, in particular different specific, stationary phases, are located between a single set of two electrodes, in particular forming stacked layers building a stack, in particular of stationary phases. Hence, solely two electrodes need to be supplied for applying the voltage for eluting any of the stationary phases and/or all stationary phases may be eluted using the same two electrodes, e.g., with all stationary phases and/or their ligands and/or receptors disposed between those two electrodes.

Alternatively, the first, in particular specific, stationary phase may be located between a first set of two electrodes and the second, in particular different specific, stationary phase may be located between a second set of two electrodes, in particular each, in particular specific, stationary phase may be located between an own set of two electrodes. Such arrangement may provide the option to apply voltage to solely one set of electrodes at a time, thereby preferably more securely solely eluting one, in particular specific, stationary phase of the at least two, in particular specific, stationary phases. In such case the first fluid is preferably sequentially brought in contact with both, first and second, stationary phases.

In yet another particularly beneficial arrangement, a set of three electrodes are provided such that the first, in particular specific, stationary phase is located between a first and a second electrode of the set of three electrodes and second, in particular specific, stationary phase is located between the second and a third electrode of the set of three electrodes. Hence, one of the electrodes is placed between the first and second, in particular specific, stationary phase, in particular shared by the first and second stationary phase. In such arrangement, voltage may be applied between two of the three electrodes which can be chosen, in particular such that an electromagnetic field is either induced/established in solely one vicinity of the stationary phases, thus either in the vicinity of the first or second stationary phase, or in the vicinities of both the first and second stationary phase. Moreover, said arrangement may be extended by further, in particular specific, stationary phases. In this regard, e.g., a third, in particular specific, stationary phase may be located between the third electrode and an additional fourth electrode. Again, the vicinities of the stationary phases in which an electromagnetic field is induced/established may be chosen, by choosing the electrodes for which voltage is applied in between for elution. Within the same concept, the method may be extended by adding further additional stationary phases exceeding three, in particular selective, stationary phases. In such case the first fluid is preferably sequentially brought in contact with the stationary phases, e.g., both, first and second, stationary phases.

Preferably, within elution step d), the voltage applied and/or the electrodes and/or sets of electrodes used for applying the voltage is/are chosen so that,
biomolecules bound to the first, in particular specific, stationary phase in step b) are eluted and biomolecules bound to the second, in particular specific, stationary phase in step b), in particular all other biomolecules bound in step b), remain bound, or
biomolecules bound to the second, in particular different specific, stationary phase in step b) are eluted and biomolecules bound to the first, in particular specific, stationary phase in step b), in particular all other biomolecules bound in step b), remain bound.

Hence, only biomolecules bound to one of the, in particular specific, stationary phases are eluted, in particular at the same time. Preferably, after elution of one of the first and second stationary phase, in particular any one of the, in particular specific, stationary phases, the eluate is removed before elution the other of the first and second, in particular specific, stationary phase, in particular any other, in particular specific, stationary phase.

In yet another advantageous embodiment, the stationary phase(s), in particular each specific stationary phase, may be formed by at least two, preferably a multitude of, especially up to 50, in particular between 5 and 20, preferably less than 10, porous substrates, in particular membranes, in particular affinity substrates, in particular membranes. In particular, this may enhance binding capacity of the, in particular specific, stationary phase(s). Moreover, the at least two porous substrates, in particular membranes, being permeable substrates, in particular membranes, may be in particular may be oriented such that, fluid may pass through the porous substrates, in particular membranes, as well as pass between the porous substrates, in particular membranes, in particular forming stacked layers. This allows for good contact between fluid and stationary phase(s).

In certain further beneficial embodiments, the at least two, in particular specific, stationary phases, in particular all specific stationary phases, and/or the at least two affinity substrates, in particular membranes, and/or specific affinity substrates, in particular membranes, and/or respectively the non-conductive carriers, in particular forming those stationary phase(s), may in particular be arranged in a stacked and/or pleated and/or wound arrangement and/or in a disjunct arrangement, with the specific stationary phases being, e.g., placed apart from another in a direction of flow of the fluid containing biomolecules, e.g., in particular passing the first fluid and/or the fluids over one after the other. Aforementioned arrangement concepts will be described further below in more detail.

Especially, every part of the first fluid is passed over every different specific stationary phase. When a multitude of identical specific stationary phases are used one might pass each part of the first fluid over every stationary phase or only some stationary phase(s).

Within a stacked arrangement, the at least two, in particular specific, in particular different specific, stationary phases, and/or the at least two affinity substrates, in particular membranes, and/or specific affinity substrates, in particular membranes, and/or respectively the non-conductive carriers are arranged as stacked layers building a stack. Preferably, the stack is formed by at least two porous substrates, in particular permeable membranes, forming stacked layers. In particular the stack is formed such that, the a first of the electrodes, in particular formed by a conductive, in particular metallic coating or metallic net on a first affinity substrate, in particular membrane, and/or first non-conductive carrier, is disposed on a first end of the stack and a second of the electrodes, in particular formed by a conductive, in particular metallic coating or metallic net on a second affinity substrate, in particular membrane, and/or second non-conductive carrier, is disposed on a second end opposite to the first end of the stack, wherein the stack comprises an affinity substrate, in particular membrane, core comprising at least one affinity substrate, in particular membrane, and/or specific affinity substrates, in particular membrane, placed between the first and second electrode, wherein the affinity substrate, in particular membrane, core in particular acts as the insulator between the first and second electrode.

In particular, the affinity substrate, in particular membrane, core comprises at least one first specific affinity substrate, in particular membrane, and at least one second specific affinity substrate, in particular membrane. Additionally, or alternatively, the affinity substrate, in particular membrane, core comprises at least two sub-cores, wherein the sub-cores are stacked within the stack, in particular forming stacked layers, the sub-cores preferably being specific sub-cores. Of those specific sub-cores a first specific sub-core may preferably comprise first specific affinity substrates, in particular membranes, and a second specific sub-core may preferably comprise second different specific affinity substrates, in particular membranes.

In particular, at least one further electrode, in particular provided as an electrically conductive, in particular metal, coating and/or metallic net disposed on at least one further non-conductive carrier, in particular formed by porous substrate(s), in particular polymeric membrane(s), and/or at least one affinity substrate, in particular membrane, of one of the sub-cores located next to an affinity substrate, in particular membrane, of one other sub-core, is placed between the at least two sub-cores and/or their ligands and/or receptors, in particular between the first and second specific sub-core. In particular, said at least one further electrode may enable to selectively apply release voltage to parts of the arrangement, respectively any single sub-core, in particular selectively inducing/establishing an electromagnetic field in the vicinity of and/or over the stationary phases and/or membranes forming the respective sub-core(s), in step d), especially, e.g., one sub-core after another.

In particular, electrode(s) may be placed between each specific stationary phase(s) and/or between layers, in particular affinity substrates, in particular membranes, of the stationary phases and/or between layers, in particular specific affinity substrates, in particular membranes, of the specific stationary phases and/or in between layers and/or stationary phases and or substrates, in particular membranes, forming sub-cores and/or their ligands and/or receptors.

With respect to a disjunct arrangement of, in particular specific, stationary phases, the at least two, in particular specific, stationary phases, are preferably placed one behind the other in terms of flow direction, in particular of the first fluid, one behind the other and/or the first fluid is passed sequentially over the at least two, in particular specific, stationary phases. Preferably, the at least two stationary phases are two different specific stationary phases.

In yet another aspect, a pleated and/or wound arrangement of stationary phase(s), in particular formed by substrate(s), in particular membrane(s), in particular stacked layers of membranes, preferably including at least two or all electrodes, is provided. In such arrangement preferably a stack of stationary phases and electrodes is used and pleated and/or wound together. Such arrangement is particular useful for chromatographic methods, in particular including but not limited to the method according to the above disclosure. In particular such arrangement may be used as part of any suitable chromatographic apparatus.

In this regard, the pleated arrangement is generated by folding a stationary phase or alternatively a stack of stationary phases, especially substrate(s), preferably membrane(s), preferably including at least two or all electrodes, into a multitude of pleats. With respect to the wound arrangement, the stationary phase or alternatively stack of stationary phases is preferably wound into a series of wound stacked layers. The pleated or wound arrangement is placed within a housing. Generally, the housing may be chosen to host the pleated and/or wound arrangement such that fluids may be passed over and/or through the stationary phase(s), in particular substrate(s), preferably membrane(s). The housing may provide for a distinct direction of flow, in particular provided by at least one inlet and at least one outlet for feeding (inlet) and removing (outlet) fluids. The pleated or wound arrangement may then be disposed in the housing such that, it blocks free passage from inlet to outlet and/or ensures any flow from the inlet to the outlet will pass through and/or over stationary phase(s), in particular the substrate(s), preferably membrane(s) at least once, preferably multiple times and/or at and/or through multiple, preferably disjunct, sections or areas of the stack and/or substrate(s), preferably membrane(s), preferably over and/or through all different specific phases. The pleated and/or wound arrangement may be e.g. disposed in the housing such that, a main extension of the arrangement is parallel, perpendicular or oblique to said distinct direction of flow.

Tube-like structures may act as housing for the arrangements. Such tube-like structures may possess base areas in any form, including but not limited to rectangular or circular base areas. Moreover, suitable housing may be in the form box-like structured housings providing inlet(s) and outlet(s) between which the stationary phase(s) are placed. The stationary phase(s) between those inlet(s) and outlet(s) may be fed laterally. In this regard, the stationary phases(s) is/are disposed such that, free passage from inlet to outlet is blocked and such that, fluid being fed through the outlets must pass through and/or, preferably and, over the stationary phase(s), in particular substrate(s), preferably membrane(s) at least once. Consequently, the inlet(s) must be oriented on a first side of the stationary phase(s) with the outlet(s) being arranged on a second site of the stationary phases(s), opposite, preferably diagonally opposite, to the first side.

However, circular housings consisting of an inner core and an outer cage with gap in between the core and the cage are preferred in other use scenarios. In this regard, the gap has a gap distance defined by the shortest distance between the core and the cage. The pleated or wound surface, in particular substrate, preferably membrane, is placed in the gap. Preferably, the inner core and outer cage are permeable to fluids and/or comprise the inlets/outlets for feeding fluid to the surface(s) and/or removing fluids/eluate. Preferably the fluid needs to pass radially through the arrangement in the gap to flow from inlet to outlet. Generally, only one of the inner core and outer cage has inlets with the other having outlets. In this regard, an inlet may describe any, in particular fluid permeable, passage for feeding, respectively an outlet any passage for removing. In particular, volume inside the core may be used for either feeding or removing.

With or without pleating and/or winding, in case stationary phases “of different kind” containing ligands/receptors unique to each respective kind are used, preferably the fluid is passed over all kind of stationary phases, particularly sequentially, and/or they might be arranged in a way that fluid passing from the inlet to the outlet is passed over all kind of stationary phases, particularly sequentially.

Preferably, porous substrate(s), in particular most preferably polymeric membrane(s), are used for forming and/or the stationary phase(s) in pleated and/or wound arrangements. Most preferably, the pleated and/or wound arrangement is/are formed by more than one porous substrate, in particular polymeric membrane. Of those porous substrates, preferably polymeric membranes, at least some or all are preferably coated with a metal coating or layer on at least one side of the membrane(s). Generally, the pleated and/or wound arrangement, preferably stack of membranes comprises at least two conductive coating or layers, preferably metal coatings.

In a particular advantageous embodiment, the pleated and/or wound arrangement consists of at least two porous substrates, preferably polymeric membranes, with a metal coating as the conductive coating or layer on one side of the respective membrane(s) forming a stack, respectively with further, in particular non-conductive porous substrates, preferably polymeric membranes, particularly establishing at least one stationary phase, between the coated membranes. Preferably the metal coatings are arranged such that at least two are placed at the outermost sides of the stack, one on each side, especially the outer sides of the outermost porous substrates, preferably polymeric membranes, within the stack, especially with respect to the flow the flow of direction within the housing. In this regard, it is especially beneficial to pleat and/or wind the membrane(s) and the conductive coating(s) together, especially the stationary phase with the electrodes together. In particular, porous substrates, preferably polymeric membranes, with metal coatings, in particular gold or platinum, and those without may be pleated and/or wound, in particular together. Preferably the wound and/or pleated stack consists of only one type of membrane, being uncoated, coated and/or having binding sited attached, e.g., differing only by coating/non-coating and/or binding sites.

In yet another advantageous embodiment, each porous substrate, preferably polymeric membrane, of the stack may have metal coating as the conductive coating or layer on one or both side(s) of the respective porous substrate(s), preferably membrane(s). Preferably, the or some porous substrate(s), preferably membrane(s), themselves are non-conductive and act as an insulator between the metal coatings or layers. In particular, voltage for elution may only be applied to selected metal coating(s) and/or layer(s) one at a time, with in particular voltage for elution being applied to at least one other metal coating and or to different pairs of metal coatings, each pair of pairs might share one metal coating.

Preferably, the stack is formed of “equal substrates, in particular membrane(s)”, hence porous substrate(s), preferably membrane(s), of the same type. Of those, at least some preferably comprise conductive coating(s) or all comprise conductive coating(s). Preferably, some of those may comprise conductive coating(s) while others preferably may not, and especially later preferably comprise binding sites and former might or might not comprise binding sites.

However, in yet another advantageous embodiment, different specific stationary phases, in particular with different ligands/receptors immobilized on the substrate, preferably membrane, in particular the ligands/receptors being unique to a single of the different substrates, preferably membranes, may be used in combination. In this regard, providing arrangements in which voltages, especially for elution, may be applied to one type of specific stationary phase disjunct from other type(s), may be beneficial. In this regard, of substrates, preferably membranes, of any type, at least one of those substrates, preferably membranes, of said type particularly has a conductive coating or layer. Between the electrically conductive coatings or layers of the types at least one insulator, especially formed by any of the porous substrate, preferably polymeric membranes, is provided. Said arrangement may be used to selectively apply release voltages to selected substrate(s), preferably membrane(s) and/or substrate(s), preferably membrane(s) of selected type.

Preferably, with respect to all embodiments of the pleated and/or wound arrangement formed by stack, the stack is pleated and/or wound as a whole, in particular all substrates, preferably membranes, and electrically conductive coatings are pleated and/or wound together.

Using a pleating or winding assistant and/or promoter may be beneficial as well, especially for achieving an easier preparation of the pleats/windings as well as maintaining a shape of the pleats/windings prepared by pleating or the stacked layers formed by winding after such pleating/winding. Such assistant(s) and/or promoter(s) may consist of one or more thermoplastic layers, which is heated during preparation of the pleats/windings, in particular to temperatures between 30° C. and 100° C. and/or above the glass transition temperature, and will retain the pleats and/or winds, especially their shapes, after said preparation, especially at temperatures below afore-mentioned temperatures. Preferably, the pleating and/or winding assistant and/or promoter is porous and/or permeable to fluids as well.

By pleating or winding the electrodes and/or stationary phase, in particular porous substrate(s), preferably membranes(s), higher surface areas per volume may be provided compared to unpleated or non-wound, hence flat electrodes and/or stationary phase, in particular substrate(s), preferably membrane(s), especially when taking into account the housing.

The surface area to volume ratio may be influenced by choosing a form of the multitude pleats, especially with respect to the housing. In this regard, the pleats consist of flat areas located between local folding points, with each flat area having a length, the pleats preferably having a M-shape as the from. For each pleat, preferably a first length is chosen to surpass at least one extension, especially width, diameter or alike, of the housing, especially, for circular housing with outer cage and inner core the distance of the gap. In this regard, due to the core having a smaller circumference compared to the cage, pleats with regular, thus at least near identical lengths of surface areas will lead to lower, at least non-optimized, surface area to volume ratios. Hence, pleats with irregular M-shape are preferred, since those forms yield higher, especially optimized surface area to volume ratios. Consequently, such irregular M-shapes comprises flat areas with at least one second length differing from the first length. Most preferably, pleat sets consisting of more than one pleat are used. Those pleats of any pleat set preferably comprises pleats with at least two different irregular M-shapes. In this regard, the at least one second length may smaller than the distance of the gap or width of the channel. Most preferably, the pleat sets are, in particular regularly repeated, within the housing. In particular, for circular housing, the pleats and/or pleat sets form a, in particular full, circle within the housing.

With respect to wound arrangements, circular housings are beneficial as well. The wounds may be formed by wrapping the surface(s) or stack of surface(s) around the inner core of the housing, thereby the inner core in particular supports the winds formed by wrapping.

Experimental section:

Hereafter, results of further experiments are presented. Within those, an affinity membrane with ligands in form of protein A immobilized on the membrane forming the binding sites was used to purify, separate, select and/or isolate the monoclonal antibody Trastuzumab. The stationary phase was provided as an arrangement consisting of the affinity membrane placed between two porous electrodes. Those electrodes were formed by metal coatings on non-conductive carries in form of other polymer membranes.

### BRIEF DESCRIPTION OF THE DRAWINGS

FIG. 1 depicts the results of an experimental procedure for determining the elution voltage needed for eluting Trastuzumab previously bound to the protein A ligands immobilized on the affinity membrane.

FIG. 2 depicts a subsequent experiment for quantitative analysis of the elution of Trastuzumab using the same experimental setup.

### DETAILED DESCRIPTION

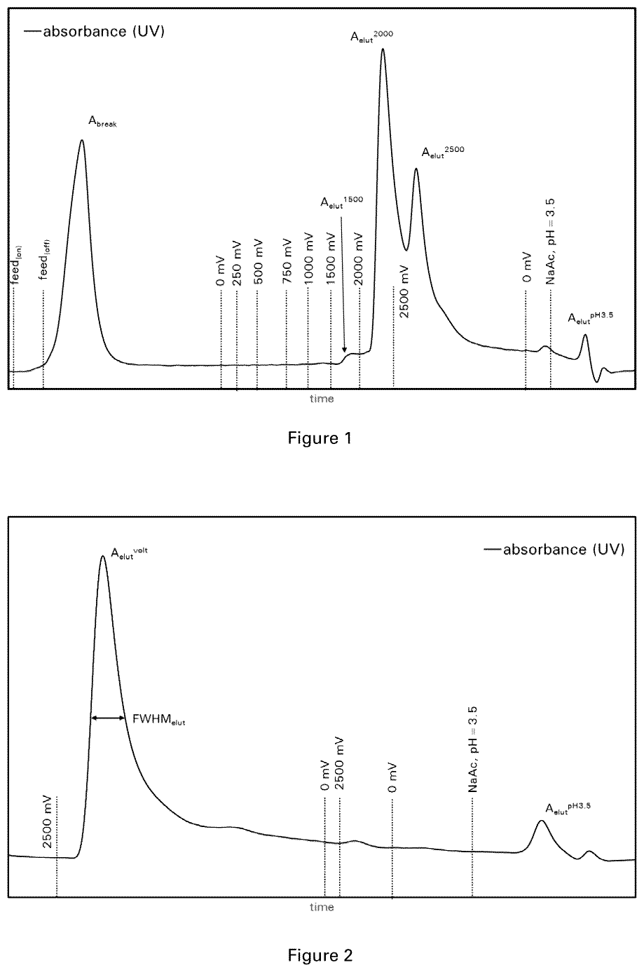

In this regard, FIG. 1 shows the absorbance (solid line) of fluids passed through the affinity membrane continuously measured over time using UV detection some short distance after passing through the membrane.

The affinity membrane was loaded/fed with a solution of Trastuzumab in ultra-pure water (starting at time feed₍ₒₙ₎ within FIG. 1). A rise in absorbance marking the breakthrough of Trastuzumab through the membrane (see start of peak A_{break} indicates the membrane to be fully loaded. Consequently, loading/feeding of the membrane with Trastuzumab was stopped at time feed_{(off)}. Subsequently, low-concentrated phosphate-saline buffer (PBS) (approx. 12 μM total phosphate, 0.15 mM sodium chloride) was continuously passed through the membrane, removing residual Trastuzumab not bound to the affinity membrane.

Afterwards, elution voltage was applied to the affinity membrane, wherein the voltage was stepwise increased (see times with respective voltages). A first minor peak Aₑₗᵤₜ¹⁵⁰⁰ was observed after a voltage of 1500 mV was applied, indicating start of elution of Trastuzumab. However, applying a voltage of 1500 mV solely induced a minor elution of Trastuzumab. After increasing said voltage to 2000 mV elution significantly increased (see absorbance peak Aₑₗᵤₜ²⁰⁰⁰). However, increasing the voltage further to 2500 mV yielded another peak in absorbance Aₑₗᵤₜ²⁵⁰⁰ showing that in order to maximize elution of Trastuzumab voltages of 2000 mV needed to be exceeded within the respective experimental setup.

Subsequently, the voltage was switched off down to zero leading to a minor increase in absorbance which may originate form Trastuzumab being temporarily adsorbed during voltage supply, in particular arguably by electro-sorption, to the electrodes to which the voltage was applied.

Afterwards a pH induced elution was performed by passing a sodium acetate (NaAc) buffer solution at pH 3.5 (1M NaAc) through the affinity membrane. Said pH elution yielded a further small peak in absorbance Aₑₗᵤₜ^{pH3.5}. Said absorbance peak may originate from Trastuzumab bound to the affinity membrane which was not eluted by applying the elution voltage but may as well originate from residual Trastuzumab which was previously adsorbed to other parts of the experimental setup, e.g., on surfaces of hoses, the membrane housing or other parts of the experimental aperture.

A subsequent experiment for quantitative analysis of the elution of Trastuzumab using the same experimental setup is depicted in FIG. 2.

The affinity membrane was again fed with Trastuzumab until the membrane was fully loaded indicated by UV detection of the fluids passed through the membrane analogously to the experiment shown within FIG. 1 (not shown in FIG. 2). The mass of Trastuzumab bound to the binding sites of the affinity membrane was calculated to be approx. 480 μg (based on the concentration of Trastuzumab in the feeding solution, the feed speed and the time needed to fully load the membrane). The used affinity membrane (effective diameter: 22 mm; thickness: 0.2 mm) possessed an effective bed volume of approx. 75 μl. Hence, the used membrane had a binding capacity of approx. 6.4 mg/cm³.

Elution was initiated by applying the elution voltage as determined within the prior experiment being 2500 mV, while continuously passing low-concentrated phosphate-saline buffer (PBS) (approx. 12 μM total phosphate, 0.15 mM sodium chloride) through the membrane. The peak Aₑₗᵤₜ^{volt} quickly arose within the UV detection with the maximum peak at approx. 13 bed volumes after applying the elution voltage (with said time including the delay of the eluted Trastuzumab leaving the vicinity of the affinity membrane and traveling to the UV spectrometer). Analyzing the elution peak in the UV, a full width at half maximum of approx. 9 bed volumes was determined (shown as FWHMₑₗᵤₜ in FIG. 2). Hence, the voltage applied initiated a fast elution yielding a narrow elution peak of only a few bed volumes. Consequently, the method described may be efficiently used for yielding concentrated biomolecule solutions.

After the elution peak dropped strongly, voltage was switched off and on. Said switching the voltage on and off, led to solely a minor increase in absorbance within the UV detection. Said additional increase may originate—analogously to the experiment in FIG. 1—from Trastuzumab temporarily bound to the electrodes, in particular by electro-sorption, after being eluted form the binding sites.

For qualitative analysis, pH elution of the affinity membrane using sodium acetate (NaAc) buffer solution at pH 3.5 (1M NaAc) was performed yielding another elution peak Aₑₗᵤₜ^{pH3.5} in the UV detection. Comparing peaks Aₑₗᵤₜ^{volt} and Aₑₗᵤₜ^{pH3.5}, in particular with respect to the areas under the curves, it can be concluded that approx. 95% of the Trastuzumab fed onto the affinity membrane were eluted by applying the elution voltage. Again, the Trastuzumab not eluted by applying the elution voltage but due to pH elution may originate from residual Trastuzumab which was previously adsorbed to other parts of the experimental setup, e.g., on surfaces of hoses, the membrane housing or other parts of the experimental aperture.

1. Method for purifying, separating, selecting and/or isolating biomolecules by means of affinity chromatography comprising the following steps:
a) providing a stationary phase, wherein the stationary phase contains, in particular selective, binding sites formed by ligands and/or receptors;
b) bringing a first fluid containing biomolecules in contact with the stationary phase, thereby binding at least parts of the biomolecules to the, in particular selective, binding sites of the stationary phase by forming a ligand/receptor complex, in particular via non-covalent interaction(s), wherein one of the biomolecules and binding sites acts as the ligand and the other of the biomolecules and binding sites acts as the receptor;
c) preferably passing at least parts of the first fluid over and/or through the stationary phase and/or optionally bringing a second fluid, in particular without biomolecules, in contact with the stationary phase;
d) eluting biomolecules previously bound in sept b);
characterized in that elution in step d) is performed by applying a voltage between two electrodes.

2. The method according to claim 1, wherein the stationary phase comprises, in particular is formed by, at least one permeable affinity substrate, in particular membrane, wherein ligands and/or receptors forming binding sites are immobilized on the at least one affinity substrate, in particular membrane, in particular on an inner and/or outer surface of the at least one affinity substrate, in particular membrane.

3. The method according to any of the preceding claims, wherein at least one of the electrodes is formed by at least one electrically conductive, in particular metal, coating and/or metallic net, wherein in particular the at least one coating and or net is formed and/or disposed on at least one non-conductive carrier, in particular formed by porous substrate(s), in particular polymeric membrane(s), and/or on the at least one affinity substrate, in particular membrane.

4. The method according to any of the preceding claims, wherein at least one of the electrodes, in particular the conductive coating and/or metallic net forming the electrode(s), consists of metal, in particular metal coating(s), preferably gold or platin, in particular consisting of a single metal, preferably gold or platin.

5. The method according to any of the preceding claims, wherein at least one of the electrodes and/or the at least one conductive coating and/or metallic net forming the electrode(s) and/or the at least one non-conductive carrier is/are permeable.

6. The method according to any of the preceding claims, wherein the stationary phase contains at least one of the, preferably at least two or all of the, electrodes, in particular formed by metal coating(s) and/or the metallic net(s) disposed on the at least one affinity substrate, in particular membrane, and/or on the at least one non-conductive carrier, wherein in particular ligands and/or receptors forming binding sites are immobilized on the at least one electrode contained in the stationary phase.

7. The method according to any of the preceding claims, wherein the at least one affinity substrate, in particular membrane and/or the at least one non-conductive carrier acts as an insulator between electrodes and/or an insulator is placed between the electrodes, in particular formed by the at least one affinity substrate, in particular membrane and/or the at least one non-conductive carrier.

8. The method according to any of the preceding claims, wherein the ligands and/or receptors are antigen ligands and/or receptors.

9. The method according to any of the preceding claims, wherein the ligands and/or receptors, in particular antigen ligands and/or receptors, are proteins or aptamers, in particular oligopeptides or oligonucleotides.

10. The method according to any of the preceding claims, wherein the ligands and/or receptors are immobilized on, in particular on surfaces, in particular outer and/or inner surfaces of, the stationary phase, in particular the at least one affinity membrane, in particular by EDC or DCC coupling and/or radical coupling, in particular through electronic beam treatment.

11. The method according to any of the preceding claims, wherein the biomolecules to be purified, separated, selected and/or isolated are proteins, in particular immunoglobulins, or antibodies, in particular, natural or monoclonal antibodies, or enzymes or nucleic acids.

12. The method according to any of the preceding claims, wherein the first fluid contains at least a first biomolecule and a second biomolecule, wherein the ligands and/or receptors forming binding sites are chosen, so that the first biomolecule has a higher binding affinity to the ligands and/or receptors compared to the second biomolecule, in particular compared to all other biomolecules contained in the first fluid, and/or so that the binding affinity of the second biomolecule, in particular all other biomolecules contained in the first fluid being different from the first biomolecule, is/are low and/or zero.

13. The method according to any of the preceding claims, wherein, especially during elution by applying a voltage and/or during a least some time during elution by applying a voltage, no elution by means of pH-value amendment is performed and/or wherein any fluid added within the elution step, especially during elution by applying a voltage and/or during a least some time during elution by applying a voltage, has a pH value equal to the first fluid and/or differ in pH value by less than 1.0, in particular less than 0.5, in particular less than 0.2, in particular less than 0.1 compared to pH value of the first fluid.

14. The method according to any of the preceding claims, wherein no fluids with salt concentrations exceeding 150 mM are added for elution of the bound biomolecules.

15. The method according to any of the preceding claims, wherein the voltage applied for elution is in the range from 0.1 to 50 volts, in particular from 0.1 to 3 volts, in particular 1.5 to 3.0 volts, in particular to 2.5 volts.

16. The method according to any of the preceding claims, wherein the stationary phase, consists of at least two specific stationary phases, each specific stationary phase in particular being formed by at least one, in particular a multitude of, specific affinity substrate(s), in particular membrane(s), wherein each specific stationary phase, in particular each specific affinity substrate, in particular membrane, contains specific binding sites formed by ligands and/or receptors, wherein the specific binding sites of a first specific stationary phase, in particular first specific affinity substrate(s), in particular membrane(s), are formed by first ligands and/or receptors and the specific binding sites of a second specific stationary phase, in particular second specific affinity substrate(s), in particular membrane(s), are formed by second ligands and/or receptors, wherein the first and second ligands and/or receptors are different.

17. The method according to claim 16, wherein the at least two specific stationary phases are arranged such that
the first and second specific stationary phase, in particular all specific stationary phases, are located between a single set of two electrodes; or
the first specific stationary phase is located between a first set of two electrodes and the second specific stationary phase is located between a second set of two electrodes; or
a set of three electrodes is provided, such that the first specific stationary phase is located between a first and a second electrode of the set of three electrodes and second specific stationary phase is located between a first and a second of the set of three electrodes.

18. The method according to claim 17, wherein the voltage applied in step d) is chosen and/or the electrodes and/or sets of electrodes used for applying the voltage are chosen, so that
a) biomolecules bound to the first stationary phase in step b) are eluted and biomolecules bound to the second stationary phase in step b), in particular all other biomolecules bound in step b), remain bound, or
b) biomolecules bound to the second stationary phase in step b) are eluted and biomolecules bound to the first stationary phase in step b), in particular all other biomolecules bound in step b), remain bound.

19. The method according to any of the preceding claims, wherein the stationary phase and/or the specific stationary phase(s) each comprise(s), in particular is/are formed by, at least two, preferably a multitude of, affinity substrates, in particular membranes and/or specific affinity substrates, in particular membranes.

20. The method according to claim 19, wherein the at least two affinity substrates, in particular membranes, and/or specific affinity substrates, in particular membranes, and/or respectively the non-conductive carriers are arranged as stacked layers building a stack.

21. Method according to claim 20, wherein the stack is formed such that, a first of the electrodes, in particular formed by a conductive, in particular metallic coating or metallic net on a first affinity substrate, in particular membrane, and/or first non-conductive carrier, is disposed on/at a first end of the stack and a second of the electrodes, in particular formed by a conductive, in particular metallic coating or metallic net on a second affinity substrate, in particular membrane, and/or second non-conductive carrier, is disposed on/at a second end opposite to the first end of the stack, wherein the stack comprises an affinity membrane core comprising at least one affinity substrate, in particular membrane, and/or specific affinity substrate, in particular membrane, placed between the first and second electrode, wherein the affinity substrate, in particular membrane, core in particular acts as the insulator between the first and second electrode.

22. Method according to claim 21, wherein the affinity substrate, in particular membrane, core comprises at least one first specific affinity substrate, in particular membrane, and at least one second specific affinity substrate, in particular membrane.

23. Method according to claim 21 or 22, wherein the affinity substrate, in particular membrane, core comprises at least two sub-cores, wherein the sub-cores are stacked within the stack and/or core.

24. Method according to claim 23, wherein the sub-cores are specific sub-cores, wherein a first specific sub-core comprises first specific affinity substrates, in particular membranes, and a second specific sub-core comprises second specific affinity substrates, in particular membranes.

25. Method according to claim 23 or 24, wherein at least one further electrode, in particular provided as an electrically conductive, in particular metal, coating and/or metallic net disposed on at least one further non-conductive carrier, in particular formed by porous substrate(s), in particular polymeric membrane(s), and/or at least one affinity substrate, in particular membrane, of one of the sub-cores located next to an affinity substrate, in particular membrane, of one other sub-core, is placed between the at least two sub-cores, in particular between the first and second specific sub-core.

26. Method according to any of the claims 16 to 24, wherein electrode(s) are placed between specific stationary phase(s) and/or between layers, in particular affinity substrates, in particular membranes, of the stationary phases and/or between layers, in particular specific affinity substrates, in particular membranes, of the specific stationary phases.

27. Method according to any of the claims wherein at least two stationary phases, in particular specific stationary phases are placed one behind the other in terms of flow direction and/or the first fluid is passed sequentially over the at least two stationary phases, in particular specific stationary phases.

28. Method according to any of the preceding claims, wherein the affinity substrate(s), in particular membrane(s), and the electrodes and/or the stack is/are pleated and/or wound, in particular together, in particular as a stack.

29. Affinity chromatography device for purifying, separating, selecting and/or isolating biomolecules comprising a stationary phase characterized in that the stationary phase comprises, in particular is formed by, at least one porous affinity substrate arranged between at least two electrodes, particularly in a stacked arrangement, wherein the at least one affinity substrate comprises at least one substrate as stationary phase with binding sites formed by ligands and/or receptors immobilized on the porous affinity substrate.

30. Device according to claim 29, wherein, the at least one porous affinity substrate is at least one membrane, in particular polymer membrane.

31. Device according to claim 29 or 30, wherein at least one of the electrodes is formed by at least one electrically conductive, in particular metal, coating and/or metallic net, wherein in particular the at least one coating and or net is formed and/or disposed on at least one non-conductive carrier, in particular formed by porous substrate(s), in particular polymeric membrane(s), and/or on the at least one affinity substrate, in particular membrane.

32. Device according to any of the claims 29 to 31, wherein the electrodes are adapted to have a voltage applied between them, preferably in the range from 0.1 to 50 volts, in particular from 0.1 to 3 volts, in particular 1.5 to 3.0 volts, in particular to 2.5 volts and/or wherein the electrodes are adapted to exhibit an electric field over the stationary phase when a voltage is applied to them.

33. Device according to any of the claims 29 to 32, wherein at least one of the electrodes, in particular the conductive coating and/or metallic net forming the electrode(s), consists of metal, in particular metal coating(s), preferably gold or platinum, in particular consisting of a single metal, preferably gold or platinum.

34. Device according to any of the claims 29 to 33, wherein at least one of the electrodes and/or the at least one conductive coating and/or metallic net forming the electrode(s) and/or the at least one non-conductive carrier is/are permeable.

35. Device according to any of the claims 29 to 34, wherein the stationary phase contains at least one of the, preferably at least two or all of the, electrodes, in particular formed by metal coating(s) and/or the metallic net(s) disposed on the at least one affinity substrate, in particular membrane, and/or on the at least one non-conductive carrier, wherein in particular ligands and/or receptors forming binding sites are immobilized on the at least one electrode contained in the stationary phase.

36. Device according to any of the claims 29 to 35, wherein the at least one affinity substrate, in particular membrane and/or the at least one non-conductive carrier acts as an insulator between electrodes and/or an insulator is placed between the electrodes, in particular formed by the at least one affinity substrate, in particular membrane and/or the at least one non-conductive carrier.

37. Device according to any of the claims 29 to 36, wherein the ligands and/or receptors are antigen ligands and/or receptors.

38. Device according to any of the claims 29 to 37, wherein the ligands and/or receptors, in particular antigen ligands and/or receptors, are proteins or aptamers, in particular oligopeptides or oligonucleotides.

39. Device according to any of the claims 29 to 38, wherein the ligands and/or receptors are immobilized on, in particular on surfaces, in particular outer and/or inner surfaces of, the stationary phase, in particular the at least one affinity membrane, in particular by EDC or DCC coupling and/or radical coupling, in particular through electronic beam treatment.

40. Device according to any of the claims 29 to 39, wherein the stationary phase, consists of at least two specific stationary phases, each specific stationary phase in particular being formed by at least one, in particular a multitude of, specific affinity substrate(s), in particular membrane(s), wherein each specific stationary phase, in particular each specific affinity substrate, in particular membrane, contains specific binding sites formed by ligands and/or receptors, wherein the specific binding sites of a first specific stationary phase, in particular first specific affinity substrate(s), in particular membrane(s), are formed by first ligands and/or receptors and the specific binding sites of a second specific stationary phase, in particular second specific affinity substrate(s), in particular membrane(s), are formed by second ligands and/or receptors, wherein the first and second ligands and/or receptors are different.

41. Device according to any of the claims 29 to 40, wherein the stationary phase and/or the specific stationary phase(s) each comprise(s), in particular is/are formed by, at least two, preferably a multitude of, affinity substrates, in particular membranes and/or specific affinity substrates, in particular membranes.

42. Device according to any of the claims 29 to 41, wherein the at least two affinity substrates, in particular membranes, and/or specific affinity substrates, in particular membranes, and/or respectively the non-conductive carriers are arranged as stacked layers building a stack.

43. Device according to claim 42, wherein the stack is formed such that, a first of the electrodes, in particular formed by a conductive, in particular metallic coating or metallic net on a first affinity substrate, in particular membrane, and/or first non-conductive carrier, is disposed on/at a first end of the stack and a second of the electrodes, in particular formed by a conductive, in particular metallic coating or metallic net on a second affinity substrate, in particular membrane, and/or second non-conductive carrier, is disposed on/at a second end opposite to the first end of the stack, wherein the stack comprises an affinity membrane core comprising at least one affinity substrate, in particular membrane, and/or specific affinity substrate, in particular membrane, placed between the first and second electrode, wherein the affinity substrate, in particular membrane, core in particular acts as the insulator between the first and second electrode.

44. Device according to claim 43, wherein the affinity substrate, in particular membrane, core comprises at least one first specific affinity substrate, in particular membrane, and at least one second specific affinity substrate, in particular membrane.

45. Device according to claim 43 or 44, wherein the affinity substrate, in particular membrane, core comprises at least two sub-cores, wherein the sub-cores are stacked within the stack and/or core.

46. Device according to claim 45, wherein the sub-cores are specific sub-cores, wherein a first specific sub-core comprises first specific affinity substrates, in particular membranes, and a second specific sub-core comprises second specific affinity substrates, in particular membranes.

47. Device according to claim 45 or 46, wherein at least one further electrode, in particular provided as an electrically conductive, in particular metal, coating and/or metallic net disposed on at least one further non-conductive carrier, in particular formed by porous substrate(s), in particular polymeric membrane(s), and/or at least one affinity substrate, in particular membrane, of one of the sub-cores located next to an affinity substrate, in particular membrane, of one other sub-core, is placed between the at least two sub-cores, in particular between the first and second specific sub-core.

48. Device according to any of the claims 29 to 47, wherein electrode(s) are placed between specific stationary phase(s) and/or between layers, in particular affinity substrates, in particular membranes, of the stationary phases and/or between layers, in particular specific affinity substrates, in particular membranes, of the specific stationary phases.

49. Device according to any of the claims 29 to 48, wherein at least two stationary phases, in particular specific stationary phases are placed one behind the other in terms of flow direction and/or the first fluid is passed sequentially over the at least two stationary phases, in particular specific stationary phases.

50. Device according to any of the claims 29 to 49, wherein the affinity substrate(s), in particular membrane(s), and the electrodes and/or the stack is/are pleated and/or wound, in particular together, in particular as a stack.
